(19) United States
(12) Patent Application Publication
(10) Pub. No.: US 2019/0085290 A1
(43) Pub. Date: Mar. 21, 2019

(54) ENHANCING THE FUNCTION IMMUNOCYTES AND HEMOCYTES USING TGP AND MICROGRAVITY

(71) Applicant: GN CORPORATION, Yamanashi (JP)

(72) Inventors: Mori YUICHI, Kanagawa (JP); Yoshioka HIROSHI, Kanagawa (JP); Terunuma HIROSHI, Tokyo (JP); Abraham SAMUEL, Chennal (IN)

(21) Appl. No.: 16/084,574

(22) PCT Filed: Mar. 14, 2017
(86) PCT No.: PCT/IB2017/051464

§ 371 Date: Sep. 13, 2018

(30) Foreign Application Priority Data
Mar. 15, 2016 (JP) 2016-051739

(51) Int. Cl. C12N 5/0783; A61K 35/17; C01B 33/152; C08F 20/56

(52) U.S. Cl. CPC C12N 5/0646; A61K 35/17; C12N 2539/10; C08F 20/56; C12N 2525/00; C01B 33/152

(57) ABSTRACT

Method of enhancing the cellular functions characterized by the inclusion of at least the process, wherein the aqueous solution containing the cells in the low temperature sol state, in an aqueous solution that exhibits thermoreversible sol-gel transition of being a sol at low temperatures and gel at high temperatures containing at least a hydrogel-forming polymer, bringing the solution containing the to a high temperature gel and then culturing the cells under microgravity.

### TECHNICAL FIELD

This invention relates to the method of enhancing the function of cells; more specifically, to the method of enhancing the function of immune cells, stem cells collected from bone marrow, umbilical cord blood or peripheral blood of a human or a predetermined animal.

### BACKGROUND ART

Generally expressed that “the universe is weightless”, however, it is not exactly “weightless”, but is referred as “microgravity”. For example, the gravitational environment in a space shuttle is about 10⁻³ G and in the space station “Kibo”, it is about 10⁻⁴ G. As the objects in space crafts and spaceships are free falling around the earth at the same speed, a pseudo “zero gravity” environment arises. A “microgravity” environment is created for 20 to 30 seconds at the top of a parabola, even in an airplane that ascends or descends in a parabola. (3D—Clinostat) is a device is a gravitational dispersion simulation microgravity device that rotates the specimen at 360° around two orthogonal axes, disperses the gravity vector in the direction of X, Y, and Z axes and cancels the gravitational vector thereby obtaining the same 10⁻³ G environment as that of space. Since ES cells and iPS cells can be cultured and maintained in an undifferentiated state (JP-A-2003-9852) by using the gravitational dispersion simulation microgravity device mentioned above, and such environment brings about various changes in cells and tissues, for example, proliferation, differentiation, activation and inhibition of cell activity, microgravity environment has gained importance in the field of regenerative medicine.

Natural killer (NK) cells are large granular lymphocytes capable of destroying cancer cells without the need of being pre-sensitized with antigen. Autoimmune Enhancement Therapy (AIET) is a method of administering a patient's own NK cells and T lymphocytes, activated by in-vitro growth culture using interleukin, to malignant tumor patients, and it is a treatment mode wherein the cytotoxicity of NK cells and T lymphocytes are exerted on the cancer cells. The special ability to kill cancer cells without the pre-sensitization of NK cells is helpful especially in attacking solid cancer and hematopoietic stem cell transplantation in in donor cell transplant; NK cells help in preventing transplant host rejection (GVHD), known to be a fatal side effect of bone marrow transplantation.

Bone marrow mononuclear cells (BMMNCs) and umbilical cord blood mononuclear cells (UCBMNCs) contain two main stem cells, namely hematopoietic stem cells (HSC) and mesenchymal stem cells (MSC). HSC plays a major role in angiogenesis, and MSC has a unique immune regulation function in extracellular matrix construction. The applications of HSC and MSC are based on their respective major biological functions of angiogenesis and extracellular matrix construction.

Nitric oxide (NO) is an important biological molecule involved in a number of physiological and histological functions of the three types of cells (NK cells, BMMNCs, UCBMNCs) mentioned above. NO is a major biological signaling molecule and plays an important role in vasodilatation, neuromodulation, vascular homeostasis and immunological reactions. NO is produced by catalytic reaction with an enzyme called nitric oxide synthase (NOS). Inducible nitric monoxide synthase (iNOS) acts in NO production, in immune reactions, and endothelial cell nitric oxide synthase (eNOS) is involved in angiogenesis. In case of NK cells, NO controls the activation of NK cells and mediates cell killing. In case of BMMNCs, NO activates eNOS of BMMNCs and enhances angiogenesis. In case of UCBMNCs, NO expresses eNOS of endothelial progenitor cells of UCBMNCs. Thus, the increase in NO promotes enhancement of the biochemical cell characteristics.

Microgravity stimulation is known to promote NO production mediated angiogenesis, via the iNOS pathway (Siamwala J H, Majumder S, Tamilarasan K P, Muley A, Reddy S H, Kolluru G K, Sinha S, Chatterjee S. Simulated microgravity promotes nitric oxide-supported angiogenesis via the iNOS-cGMP-PKG pathway in macrovascular endothelial cells. FEBS Lett. 2010 Aug. 4; 584 (15): 3415-23). In case of NK cells, space flight (Buravkova L B, Grigor'eva O V, Rykova M P, Grigor'ev A I. Cytotoxic activity of natural killer cells in vitro under microgravity Dokl Biol Sci. 2008 July-August; 421: 275-7)) and microgravity reduces the cytotoxicity of NK cells, however, it is also known that this effect is neutralized by interleukin-15 alone, or with interleukin-12. (Li Q, Mei Q, Huyan T, Xie L, Che S, Yang H, Zhang M, Huang Q. Effects of simulated microgravity on primary human NK cells. Astrobiology. 2013 August; 13 (8): 703-14). Interleukin is also used in cell activation in AIET and in vivo.

When bone marrow mesenchymal cells are cultured under microgravity, the undifferentiated state is maintained while maintaining the ability of high migration. Good results are seen in transplantation in spinal cord injury compared to the normal culture (Mitsuhara T, Takeda M, Yamaguchi S, Manabe T, Matsumoto M, Kawahara Y, Yuge L, Kurisu K. Simulated microgravity facilitates cell migration and neuroprotection after bone marrow stromal cell transplantation in spinal cord injury. Stem Cell Res Ther. 2013 Apr. 1; 4 (2): 35). When hematopoietic stem cells were cultured under microgravity, an increase in cell population and the progenitor cell population was observed compared to the normal gravity culture (Plett P A, Frankovitz S M, Abonour R, Orschell-Traycoff C M. Proliferation of human hematopoietic bone marrow cells in simulated microgravity. In vitro Cell Dev Biol Anim. 2001 February; 37 (2): 73-8).

### PRIOR ART DOCUMENT

### Non-Patent Literature

[Non-patent literature 1] Siamwala J H, Majumder S, Tamilarasan K P, Muley A, Reddy S H, Kolluru G K, Sinha S, Chatterjee S. Simulated microgravity promotes nitric oxide-supported angiogenesis via the iNOS-cGMP-PKG pathway in macrovascular endothelial cells. FEBS Lett. 2010 Aug. 4; 584 (15): 3415-23)
[Non-patent literature 2] Buravkova L B, Grigor'eva O V, Rykova M P, Grigor'ev A I. Cytotoxic activity of natural killer cells in vitro under microgravity Dokl Biol Sci. 2008 July-August; 421:275-7)
[Non-patent literature 3] Li Q, Mei Q, Huyan T, Xie L, Che S, Yang H, Zhang M, Huang Q. Effects of simulated microgravity on primary human NK cells. Astrobiology. 2013 August; 13 (8): 703-14
[Non-patent literature 4] Mitsuhara T, Takeda M, Yamaguchi S, Manabe T, Matsumoto M, Kawahara Y, Yuge L, Kurisu K. Simulated microgravity facilitates cell migration and neuroprotection after bone marrow stromal cell transplantation in spinal cord injury. Stem Cell Res Ther. 2013 Apr. 1; 4 (2): 35
[Non-patent literature 5] Plett P A, Frankovitz S M, Abonour R, Orschell-Traycoff C M. Proliferation of human hematopoietic bone marrow cells in simulated microgravity. In vitro Cell Dev Biol Anim. 2001 February; 37 (2): 73-8

### SUMMARY OF INVENTION

### Problem to be Solved by the Invention

However, simply enhancing the function of various cells by merely culturing them under microgravity was not enough. This invention is intended to provide a method for further enhancing the functions of various cells, such as the ability of NK cells to produce NO.

### Procedure of Solving the Problem

It was observed by the inventors that the ability of NK cells in the human body to kill cancer is enhanced, when an aqueous solution containing the cells in the sol state at low temperature, is embedded in aqueous solution that exhibits thermoreversible sol-gel transition of being a sol at low temperatures and gel at high temperatures containing at least a hydrogel-forming polymer, bringing the solution containing the cells to a high temperature gel and then subjecting it to microgravity for a short duration of time. From the analysis using flow cytometry, an increase in cell population and CD marker positive rate have been observed. Similarly, the same increase as mentioned above, was observed in umbilical cord blood cells and bone marrow cells. The above mentioned enhancement of cellular capacity in such cells has substantial utility in the treatment of solid organ cancer and in the treating several hemotological malignancies, in treating patients who have undergone bone marrow transplantation, etc. This cell regeneration method can be very effectively used in in regenerative medicine for recovering, reviving and restoring cells.

### Effect of Invention

As explained above, from this invention, it is possible to efficiently enhance the functions of NK cells, umbilical cord blood cells and bone marrow cells, and which has be of tremendous use in the treatment of solid organ cancer, in the treatment of several hematological malignancies and in treatment of patients who have undergone bone marrow transplantation. This regeneration method can be used very effectively as a method for recovering, reviving and restoring cells in regenerative medicine.

### Form of Implementing the Invention

The contents of this invention are explained in detail below.

Space environment or environment of an airplane ascending and descending in a parabola may also be used as the microgravity environment of this invention, however, from the ease of being carried out on the ground, the use of gravity distributed simulation microgravity device (3D—Clinostat) is desirable.

The gravity dispersed simulation microgravity device creates the same 10⁻³ G environment as that of space, by rotating the specimen at 360° and dispersing the gravitational vector in the axial direction of X, Y, and Z, and canceling the gravitational vector.

### (Hydrogel-Forming Polymer, TGP)

The hydrogel-forming polymer constituting the hydrogel according to the present invention (Thermoreversible Gelation Polymer, TGP) refers to a polymer which has a crosslinking or network structure, and has a property such that it can form a hydrogel thermoreversibly by retaining water (in the inside thereof) on the basis of such a structure. Further, the “hydrogel” refers to a gel, which comprises, at least a crosslinked or network structure comprising a polymer, and water (as a dispersion liquid) supported or retained by such a structure.

### (Sol-Gel Transition Temperature)

In the present invention, the definition and measurement of the “sol state,” “gel state,” and “sol-gel transition temperature” may also be carried out as mentioned below according to the definition and method described in a publication (H. Yoshioka et al., Journal of Macromolecular Science, A31(1), 113 (1994)). That is, the dynamic elastic modulus of a sample at an observed frequency of 1 Hz is determined by gradually shifting the temperature from a low temperature side to a high temperature side (1° C./1 min). In this measurement, the sol-gel transition temperature is defined as a temperature at which the storage elastic modulus (G′, elastic term) of the sample exceeds the loss elastic modulus (G″, viscous term). In general, the sol state is defined as a state in which G″>G′ is satisfied, and the gel state is defined as a state in which G″<G′ is satisfied. For the measurement of such a sol-gel transition temperature, the following measuring conditions can preferably be used.

### <Measuring Conditions for Dynamic and Loss Elastic Moduli>

Measuring apparatus (trade name): Stress controlled-type rheometer (model: AR-500, mfd. by TA Instruments Co.)

Concentration of sample solution (or dispersed liquid) (as a concentration of a “polymer compound having a sol-gel transition temperature”): 10% (by weight)

Amount of sample solution: about 0.8 g

Shape and size of cell for measurement: acrylic parallel disk (diameter: 4.0 cm), gap: 600 μm

Measurement frequency: 1 Hz

Stress to be applied: within linear region

In the present invention, the above sol-gel transition temperature may preferably be higher than 0° C. and not higher than 37° C., more preferably, higher than 5° C. and not higher than 35° C. (particularly not lower than 10° C. and not higher than 33° C.).

The hydrogel material having such a preferred sol-gel transition temperature may easily be selected from specific compounds as described below, according to the above-mentioned screening method (method of measuring the sol-gel transition temperature).

The hydrogel-forming polymer usable for the present invention is not particularly limited, as long as the polymer exhibits the above-mentioned thermo-reversible sol-gel transition (that is, as long as it has a sol-gel transition temperature).

As specific examples of the polymer such that an aqueous solution thereof has a sol-gel transition temperature, and it reversibly assumes a sol state at a temperature lower than the sol-gel transition temperature, there have been known, e.g., polyalkylene-oxide block copolymer represented by block copolymers comprising polypropylene oxide portions and polyethylene oxide portions; etherified (or ether group-containing) celluloses such as methyl cellulose and hydroxypropyl cellulose; chitosan derivatives (K. R. Holme. et al. Macromolecules, 24, 3828 (1991)), etc.

### (Preferred Hydrogel-Forming Polymers)

The hydrogel-forming polymer preferably usable as the polymer according to the present invention may preferably comprise a combination of plural hydrophobic blocks having a cloud point, and a hydrophilic block bonded thereto.

The presence of the hydrophilic block is preferred in view of the provision of the water-solubility of the hydrogel material at a temperature lower than the sol-gel transition temperature. The presence of the plural hydrophobic block having a cloud point is preferred in view of the conversion of the hydrogel material into a gel state at a temperature higher than the sol-gel transition temperature.

In other words, the blocks having a cloud point become water-soluble at a temperature lower than the cloud point, and are converted into a water-insoluble state at a temperature higher than the cloud point, and therefore these blocks function as crosslinking points constituted by hydrophobic bonds for forming a gel at a temperature higher than the cloud point. That is, the cloud point based on the hydrophobic bonds corresponds to the above-mentioned sol-gel transition temperature of the hydrogel.

However, it is not always necessary that the cloud point corresponds to the sol-gel transition temperature. This is because the cloud point of the above-mentioned “blocks having a cloud point” is generally influenced by the bonding between the hydrophilic block and the blocks having a cloud point.

The hydrogel to be use in the present invention utilizes a property of hydrophobic bonds such that they are not only strengthened along with an increase in temperature, but also the change in the hydrophobic bond strength is reversible with respect to the temperature. In view of the formation of plural crosslinking points in one molecule, and the formation of a gel having a good stability, the hydrogel-forming polymer may preferably have a plurality of “blocks having cloud point”.

On the other hand, as described above, the hydrophilic block in the hydrogel-forming polymer has a function of causing the hydrogel-forming polymer to be changed into a water-soluble state at a temperature lower than sol-gel transition temperature. The hydrophilic block also has a function of providing the state of an aqueous (or water-containing) gel, while preventing the aggregation and precipitation of the hydrogel material due to an excess increase in the hydrophobic binding force at a temperature higher than the transition temperature.

### (Plural Blocks Having Cloud Point)

The plural block having a cloud point may preferably comprise a polymer block which shows a negative solubility-temperature coefficient with respect to water. More specifically, such a polymer may preferably be one selected from the group consisting of: polypropylene oxide, copolymers comprising propylene oxide and another alkylene oxide, poly N-substituted acrylamide derivatives, poly N-substituted methacrylamide derivatives, copolymers comprising an N-substituted acrylamide derivative and an N-substituted methacrylamide derivative, polyvinyl methyl ether, and partially-acetylated product of polyvinyl alcohol.

In order to prepare a block having a cloud point which is decomposed and absorbed in a living body, it is effective to use a polypeptide comprising a hydrophobic amino acid and a hydrophilic amino acid, as the block having a cloud point. Alternatively, a polyester-type biodegradable polymer such as polylactic acid or polyglycolic acid can also be used as a block having a cloud point which is decomposed and absorbed in a living body.

It is preferred that the above polymer (block having a cloud point) has a cloud point of higher than 4° C. and not higher than 40° C., in view of the provision of a polymer (compound comprising a plurality of blocks having a cloud point, and a hydrophilic block bonded thereto) to be used in the present invention having a sol-gel transition temperature of higher than 4° C. and not higher than 37° C.

It is possible to measure the cloud point, e.g., by the following method. That is, an about 1 wt. %-aqueous solution of the above polymer (block having a cloud point) is cooled to be converted into a transparent homogeneous solution, and thereafter the temperature of the solution is gradually increased (temperature increasing rate: about 1° C./min.), and the point at which the solution first shows a cloudy appearance is defined as the cloud point.

Specific examples of the poly N-substituted acrylamide derivatives and poly N-substituted methacrylamide derivatives are described below.

Poly-N-acryloyl piperidine

Poly-N-n-propyl methacrylamide

Poly-N-isopropyl acrylamide

Poly-N,N-diethyl acrylamide

Poly-N-isopropyl methacrylamide

Poly-N-cyclopropyl acrylamide

Poly-N-acryloyl pyrrolidine

Poly-N,N-ethyl methyl acrylamide

Poly-N-cyclopropyl methacrylamide

Poly-N-ethyl acrylamide

The above polymer may be either a homopolymer or a copolymer comprising a monomer constituting the above polymer and “another monomer”. The “another monomer” to be used for such a purpose may be either a hydrophilic monomer, or a hydrophobic monomer. In general, when copolymerization with a hydrophilic monomer is conducted, the resultant cloud point may be increased. On the other hand, when copolymerization with a hydrophobic monomer is conducted, the resultant cloud point may be decreased. Accordingly, a polymer having a desired cloud point (e.g., a cloud point of higher than 4° C. and not higher than 40° C.) may also be obtained by selecting such a monomer to be used for the copolymerization.

### (Hydrophilic Monomer)

Specific examples of the above hydrophilic monomer may include: N-vinyl pyrrolidone, vinyl pyridine, acrylamide, methacrylamide, N-methyl acrylamide, hydroxyethyl methacrylate, hydroxyethyl acrylate, hydroxymethyl methacrylate, hydroxymethyl acrylate,

methacrylic acid and acrylic acid having an acidic group, and salts of these acids, vinyl sulfonic acid, styrenesulfonic acid, etc., and derivatives having a basic group such as N,N-dimethylaminoethyl methacrylate, N,N-diethylaminoethyl methacrylate, N,N-dimethylaminopropyl acrylamide, salts of these derivatives, etc. However, the hydrophilic monomer to be usable in the present invention is not restricted to these specific examples.

### (Hydrophobic Monomer)

On the other hand, specific examples of the above hydrophobic monomer may include: acrylate derivatives and methacrylate derivatives such as ethyl acrylate, methyl methacrylate, and glycidyl methacrylate; N-substituted alkyl methacrylamide derivatives such as N-n-butyl methacrylamide; vinyl chloride, acrylonitrile, styrene, vinyl acetate, etc. However, the hydrophobic monomer to be usable in the present invention is not restricted to these specific examples.

### (Hydrophilic Block)

On the other hand, specific examples of the hydrophilic block to be combined with (or bonded to) the above-mentioned block having a cloud point may include: methyl cellulose, dextran, polyethylene oxide, polyvinyl alcohol, poly N-vinyl pyrrolidone, polyvinyl pyridine, polyacrylamide, polymethacrylamide, poly N-methyl acrylamide, polyhydroxymethyl acrylate, polyacrylic acid, polymethacrylic acid, polyvinyl sulfonic acid, polystyrene sulfonic acid, and salts of these acids; poly N,N-dimethylaminoethyl methacrylate, poly N,N-diethylaminoethyl methacrylate, poly N,N-dimethylaminopropyl acrylamide, and salts of these, etc.

The process for combining the above block having a cloud point with the hydrophilic block is not particularly limited. For example, it is preferred to obtain a block copolymer, or a graft copolymer, or a dendrimer-type copolymer containing these blocks.

It is also possible to conduct such a combination by introducing a polymerizable functional group (such as acryloyl group) into either one of the above blocks, and copolymerizing with the resultant product a monomer capable of providing the other block.

Alternatively, it is also possible to obtain a combination product of the above block having a cloud point with the hydrophilic block by copolymerizing a monomer capable of providing the block having a cloud point with a monomer capable of providing the hydrophilic block.

In addition, the block having a cloud point and the hydrophilic block may also be combined or bonded with each other by preliminarily introducing reactive functional groups (such as hydroxyl group, amino group, carboxyl group, and isocyanate group) into both kinds of the blocks, and combining these blocks by using a chemical reaction. At this time, it is usual to introduce a plurality of reactive functional groups into the hydrophilic block.

Further, the polypropylene oxide having a cloud point and the hydrophilic block may be combined or bonded with each other by repetitively subjecting polypropylene oxide and a monomer constituting the above “other water-soluble block” (such as ethylene oxide) to a stepwise or consecutive polymerization, to thereby obtain a block copolymer comprising polypropylene oxide and a water-soluble block (such as polyethylene oxide) combined therewith.

Such a block copolymer may also be obtained by introducing a polymerizable group (such as acryloyl group) into the terminal of polypropylene oxide, and then copolymerizing therewith a monomer constituting the hydrophilic block.

Further, a polymer usable in the present invention may be obtained by introducing a functional group which is reactive in a bond-forming reaction with the terminal functional group of polypropylene oxide (such as hydroxyl group) into a hydrophilic block, and reacting the resultant hydrophilic block and the polypropylene oxide. In addition, a hydrogel-forming polymer usable in the present invention may be obtained by connecting materials such as one comprising polypropylene glycol and polyethylene glycol bonded to both terminals thereof (such as Pluronic F-127; trade name, mfd. by Asahi Denka Kogyo K.K.).

In an embodiment of the present invention wherein the hydrogel-forming polymer comprises a block having a cloud point, at a temperature lower than the cloud point, the polymer may completely be dissolved in water so as to assume a sol state, since the above-mentioned “block having a cloud point” present in the polymer molecule is water-soluble together with the hydrophilic block. However, when a solution of the above polymer is heated up to a temperature higher than the cloud point, the “block having a cloud point” present in the polymer molecule becomes hydrophobic so that separate molecules of the polymer are associated or aggregated with each other due to a hydrophobic interaction.

On the other hand, the hydrophilic block is water-soluble even at this time (i.e., even when heated up to a temperature higher than the cloud point), and therefore, the polymer according to the present invention in water is formed into a hydrogel having a three-dimensional network structure wherein hydrophobic association portions between the blocks having a cloud point constitute the crosslinking points. The resultant hydrogel is again cooled to a temperature lower than the cloud point of the “block having a cloud point” present in the polymer molecule, the block having a cloud point becomes water-soluble and the above crosslinking points due to the hydrophobic association are released or liberated so that the hydrogel structure disappears, whereby the polymer according to the present invention is again formed into a complete aqueous solution. In the above-described manner, the sol-gel transition in the polymer according to the present invention is based on the reversible hydrophilic-hydrophobic conversion in the block having a cloud point present in the polymer molecule at the cloud point, and therefore the transition has a complete reversibility in accordance with a temperature change.

### (Solubility of Gel)

As described above, the hydrogel-forming polymer according to the present invention comprising at least a polymer having a sol-gel transition temperature in an aqueous solution thereof, substantially shows a water insolubility at a temperature (d ° C.) higher than the sol-gel transition temperature, and reversibly shows water solubility at a temperature (e ° C.) lower than the sol-gel transition temperature.

The above-mentioned temperature (d ° C.) may preferably be a temperature which is at least 1° C., more preferably at least 2° C. (particularly preferably, at least 5° C.) higher than the sol-gel transition temperature. Further, the above-mentioned “substantial water insolubility” may preferably be a state wherein the amount of the above polymer to be dissolved in 100 ml of water at the above temperature (d ° C.) is 5.0 g or less (more preferably 0.5 g or less, particularly preferably 0.1 g or less).

On the other hand, the above-mentioned temperature (e ° C.) may preferably be a temperature which is at least 1° C., more preferably at least 2° C. (particularly preferably, at least 5° C.) lower than the sol-gel transition temperature, in terms of the absolute values of these temperatures. Further, the above-mentioned “water solubility” may preferably be a state wherein the amount of the above polymer to be dissolved in 100 ml of water at the above temperature (e ° C.) is 0.5 g or more (more preferably 1.0 g or more). The above “to show a reversible water solubility” refers to a state wherein an aqueous solution of the above hydrogel-forming polymer shows the above-mentioned water solubility at a temperature lower than the sol-gel transition temperature, even after the polymer is once formed into a gel state (at a temperature higher than the sol-gel transition temperature).

A 10%-aqueous solution of the above polymer may preferably show a viscosity of 10-3,000 Pa·s (10-3,000 centipoises), more preferably, 50-1,000 Pa·s (50-1,000 centipoises) at 5° C. Such a viscosity may preferably be measured, e.g., under the following measurement conditions:

Viscometer: Stress-controlled type rheometer (model: AR-500, mfd. by TA Instruments Co., USA)

Rotor diameter: 60 mm

Rotor configuration: Parallel-plate type

Measurement frequency: 1 Hz (hertz)

Even when the an aqueous solution of the hydrogel-forming polymer according to the present invention is formed into a gel state at a temperature higher than the sol-gel transition temperature, and thereafter the resultant gel is immersed in a large amount of water, the gel is not substantially dissolved in water. For example, such a characteristic of the above carrier may be confirmed in the following manner.

More specifically, 0.15 g of the hydrogel-forming polymer according to the present invention is dissolved in 1.35 g of distilled water at a temperature lower than the above sol-gel transition temperature (e.g., under cooling with ice) to thereby prepare a 10 mass %-aqueous solution. Then, the resultant solution is poured into a plastic Petri dish having a diameter of 35 mm, then the dish is warmed up to a temperature of 37° C. to form a gel having a thickness of about 1.5 mm in the dish, and the total weight of the Petri dish (f gram) containing the gel is measured. Then, the entirety of the Petri dish containing the gel is left standing in 250 ml of water at 37° C. for 10 hours, and thereafter the total weight of the Petri dish (g gram) containing the gel is measured, to thereby determine whether the gel has been dissolved from the gel surface or not. At this time, in the hydrogel-forming polymer according to the present invention, the ratio of weight decrease in the gel, i.e., the value of {(f−g)/f} may preferably be 5.0% or less, more preferably 1.0% or less (particularly preferably 0.1% or less).

Even when an aqueous solution of the hydrogel-forming polymer according to the present invention was converted into a gel state at a temperature higher than the sol-gel transition temperature, and then the resultant gel was immersed in a large amount (about 0.1-100 times larger than the gel, by volume ratio), the gel was not dissolved for a long period of time. Such a property of the polymer to be used in the present invention may be achieved, e.g., by the presence of at least two (a plurality of) blocks having a cloud point in the polymer molecule.

On the contrary, according to the present inventors' experiments, in a case where a similar gel was formed by using the above-mentioned Pluronic F-127 comprising polypropylene oxide and polyethylene oxide bonded to both terminals thereof, the resultant gel was completely dissolved when the gel is left standing in water for several hours.

In order to suppress the cytotoxicity of a non-gel state to a low level as completely as possible, it is preferred to use a hydrogel-forming polymer which can be converted into a gel state at a concentration of 20% or less (more preferably 15% or less, particularly 10% or less) in terms of the concentration of the polymer based on water, i.e., {(polymer)/(polymer+water)}×100(%).

In the present invention, the molecular weight of TGP may preferably be higher than 30,000 and not higher than 30,000,000, more preferably, higher than 100,000 and not higher than 10,000,000 (particularly not lower than 500,000 and not higher than 5,000,000).

### EXAMPLES

Herein below, the present invention will be described in more detail with reference to Examples. However, it should be noted that the present invention is defined by Claims, but is not limited by the following Examples.

### Production Example 1

10 g of a polypropylene oxide-polyethylene oxide copolymer (average polymerization degree of propylene oxide/ethylene oxide=about 60, Pluronic F-127, mfd. by Asahi Denka K.K.) was dissolved in 30 ml of dry chloroform, and in the co-presence of phosphorus pentaoxide, 0.13 g of hexamethylene diisocyanate was added thereto, and the resultant mixture was subjected to reaction under refluxing at the boiling point for six hours. The solvent was distilled off under reduced pressure, the resultant residue was dissolved in distilled water, and subjected to ultrafiltration by using an ultrafiltration membrane having a molecular cutoff of 3×10⁴ (Amicon PM-30) so as to fractionate the product into a low-molecular weight polymer fraction and a high-molecular weight polymer fraction. The resultant aqueous solution was frozen, to thereby obtain a high-polymerization degree product of F-127 and a low-polymerization degree product of F-127.

When the above high-polymerization degree product of F-127 (TGP-1, a hydrogel-forming polymer according to the present invention) was dissolved in distilled water under ice-cooling in an amount of 8 mass %. When the resultant aqueous solution was gradually warmed, it was found that the viscosity was gradually increased from 21° C., and was solidified at about 27° C. so as to be converted into a hydrogel state. When the resultant hydrogel was cooled, it was returned to an aqueous solution at 21° C. Such a conversion was reversibly and repetitively observed. On the other hand, a solution which had been obtained by dissolving the above low-polymerization degree product of F-127 in distilled water under ice-cooling in an amount of 8 mass %, was not converted into a gel state at all even when it was heated to 60° C. or higher.

### Production Example 2

160 mol of ethylene oxide was subjected to an addition reaction with 1 mol of trimethylol propane by cationic polymerization, to thereby obtain polyethylene oxide triol having an average molecular weight of about 7000.

100 g of the thus obtained polyethyleneoxide triol was dissolved in 1000 ml of distilled water, and then 12 g of potassium permanganate was slowly added thereto at room temperature, and the resultant mixture was subjected to an oxidization reaction at this temperature for about one hour. The resultant solid content was removed by filtration, and the product was subjected to extraction with chloroform, and the solvent (chloroform) was distilled off, to thereby obtain 90 g of a polyethylene oxide tricarboxyl derivative.

10 g of the thus obtained polyethylene oxide tricarboxyl derivative, and 10 g of polypropylene oxide diamino derivative (average propylene oxide polymerization degree: about 65, trade name: Jeffamine D-4000, mfd. by Jefferson Chemical Co., U.S.A., cloud point: about 9° C.) were dissolved in 1000 ml of carbon tetrachloride, and then 1.2 g of dicyclohexyl carbodiimide was added thereto, and the resultant mixture was allowed to cause a reaction for 6 hours under refluxing at boiling point. The resultant reaction mixture was cooled and the solid content was removed by filtration, and thereafter the solvent (carbon tetrachloride) therein was distilled off under reduced pressure. Then, the resultant residue was dried under vacuum, to thereby obtain a polymer for coating (TGP-2) comprising plural polypropylene oxide blocks, and polyethylene oxide block combined therewith. This polymer was dissolved in distilled water under cooling with ice so as to provide a concentration of 5 mass %. When the sol-gel transition temperature of the resultant aqueous solution was measured, it was found that the sol-gel transition temperature was about 16° C.

### Production Example 3

96 g of N-isopropyl acrylamide (mfd. by Eastman Kodak Co.), 17 g of N-aclyloxy succinimide (mfd. by Kokusan Kagaku K.K.), and 7 g of n-butyl methacrylate (mfd. by Kanto Kagaku K.K.) were dissolved in 4000 ml of chloroform. After the purging with nitrogen gas, 1.5 g of N,N′-azobisisobutyronitrile was added thereto, and the resultant mixture was subjected to polymerization at 60° C. for 6 hours. The reaction mixture was concentrated, and then was reprecipitated in diethyl ether. The resultant solid content was recovered by filtration, and then was dried under vacuum, to thereby obtain 78 g of poly (N-isopropyl acrylamide-co-N-aclyloxy succinimide-co-n-butyl methacrylate).

Then, an excess of isopropylamine was added to the thus obtained poly(N-isopropyl acrylamide-co-N-aclyloxy succinimide-co-n-butyl methacrylate) to thereby obtain poly(N-isopropyl acrylamide-co-n-butyl methacrylate). The thus obtained poly(N-isopropyl acrylamide-co-n-butyl methacrylate) had a sol-gel transition temperature of about 19° C. in its aqueous solution.

Then, 10 g of the thus obtained poly(N-isopropyl acrylamide-co-N-aclyloxy succinimide-co-n-butyl methacrylate) and 5 g of both terminal-aminated polyethylene oxide (molecular weight=6000, mfd. by Kawaken Fine Chemical K.K.) were dissolved in 1000 ml of chloroform, and the resultant mixture was allowed to cause a reaction at 50° C. for 3 hours. The reaction mixture was cooled to room temperature, and thereafter 1 g of isopropylamine was added thereto, and was left standing for 1 hour. The reaction mixture was concentrated, and then was precipitated in diethyl ether. The solid content was recovered by filtration, and thereafter was dried under vacuum, to thereby obtain a polymer for coating (TGP-3) comprising plural poly(N-isopropyl acrylamide-co-n-butyl methacrylate) blocks and polyethylene oxide block combined therewith.

This polymer was dissolved in distilled water under cooling with ice so as to provide a concentration of 5 mass %. When the sol-gel transition temperature of the resultant aqueous solution was measured, it was found that the sol-gel transition temperature was about 21° C.

### Production Example 4

### (Sterilization Method)

2.0 g of the above-mentioned polymer (TGP-1) was placed in an EOG (ethylene oxide gas) sterilizing bag (trade name: Hybrid Sterilization bag, mfd. by Hogi Medical Co.), and was filled up with EOG by use of an EOG sterilizing device (trade name: Easy Pack, mfd. Inouchi Seieido Co.) and then the bag was left standing at room temperature for twenty-four hours. Further, the bag was left standing at 40° C. for half a day, EOG was removed from the bag and the bag was subjected to aeration. The bag was placed in a vacuum drying chamber (40° C.) and was left standing for half a day, and was sterilized while the bag was sometimes subjected to aeration.

Separately, it was confirmed that the sol-gel transition temperature of the polymer was not changed even after this sterilization operation.

### Production Example 5

37 g of N-isopropylacrylamide, 3 g of n-butyl methacrylate, and 28 g of polyethylene oxide monoacrylate (having a molecular weight of 4,000, PME-4000 mfd. by Nihon Yushi K.K. (NOF Corporation)) were dissolved in 340 mL of benzene. Thereafter, 0.8 g of 2,2′-azobisisobutyronitrile was added to the resultant solution, and then was subjected to a reaction at 60° C. for 6 hours. 600 mL of chloroform was added to the thus obtained reaction product so as to be dissolved therein, and the resultant solution was dropped into 20 L (liter) of ether so as to be precipitated therein. The resultant precipitate was recovered by filtration, and the precipitate was then subjected to vacuum drying at about 40° C. for 24 hours. Thereafter, the resultant product was again dissolved in 6 L of distilled water. The solution was concentrated to a volume of 2 L at 10° C. by using a hollow fiber ultrafiltration membrane with a molecular weight cutoff of 10×10⁴ (HIP100-43 mfd. by Amicon).

The concentrated solution was diluted with 4 L of distilled water, and then, the dilution operation was carried out again. The above dilution and concentration by ultrafiltration were further repeated 5 times, so as to eliminate products having a molecular weight of 10×10⁴ or lower. The product which had not been filtrated by this ultrafiltration (i.e., the product remaining in the inside of the ultrafiltration membrane) was recovered and freeze-dried, so as to obtain 60 g of a hydrogel-forming polymer (TGP-4) according to the present invention having a molecular weight of 10×10⁴ or higher.

1 g of the thus obtained hydrogel-forming polymer (TGP-4) according to the present invention was dissolved in 9 g of distilled water under ice cooling. When the sol-gel transition temperature of the obtained aqueous solution was measured, it was found to be 25° C.

### Production Example 6

The hydrogel-forming polymer (TGP-3) according to the present invention which had been obtained in Production Example 3 was dissolved so as to provide a concentration of 10 mass % in distilled water. When the steady flow viscosity η thereof at 37° C. was measured, it was found to be 5.8×10⁵ Pa·sec. In the measurement of the steady flow viscosity q, a stress rheometer (AR-500), and an acryl disk (diameter: 4 cm) as a measuring device were used. The thickness of a sample was set to 600 μm, and applying a shearing stress of 10 N/m², the resultant creep was measured for 5 minutes after 5 minutes had passed.

On the other hand, agar was dissolved so as to provide a concentration of 2 mass % in distilled water at 90° C., and the mixed solution was converted into a gel state at 10° C. for 1 hour. Thereafter, η thereof at 37° C. was measured. As a result, the obtained value exceeded the measurement limit (1×10⁷ Pa·sec) of the apparatus.

### Production Example 7

71.0 g of N-isopropylacrylamide and 4.4 g of n-butyl methacrylate were dissolved in 1,117 g of ethanol. To the resultant mixture solution, an aqueous solution which had been obtained by dissolving 22.6 g of polyethylene glycol dimethacrylate (PDE 6000, mfd. by NOF Corporation) in 773 g of water was added. The oresultant solution was heated to 70° C. under a nitrogen stream. While the solution was maintaining at 70° C. under a nitrogen stream, 0.8 mL of N,N,N′,N′-tetramethylethylenediamine (TEMED) and 8 mL of 10% ammonium persulfate (APS) aqueous solution were added to the solution, and then was subjected to a reaction for 30 minutes under stirring. Further, 0.8 mL of TEMED and 8 mL of 10% APS aqueous solution were added thereto 4 times at 30-minute intervals, and the polymerization reaction was terminated. The reaction mixture was cooled to 10° C. or lower, it was diluted with 5 L of cold distilled water with a temperature of 10° C. Thereafter, the solution was concentrated to 2 L at 10° C., by using an ultrafiltration membrane with a molecular weight cutoff of 10×10⁴.

4 L of cold distilled water was added to the concentrated solution for dilution, and the above concentration operation using the ultrafiltration was conducted again. Thereafter, the above dilution and ultrafiltration concentration were repeated 5 times, so as to eliminate products with a molecular weight of 10×10⁴ or lower. The product which had not been filtrated by the above ultrafiltration (product remaining in the ultrafiltration membrane) was recovered and freeze-dried, so as to obtain 72 g of the hydrogel-forming polymer (TGP-5) according to the present invention with a molecular weight of 10×10⁴ or higher.

1 g of the thus obtained hydrogel-forming polymer (TGP-5) according to the present invention was dissolved in 9 g of distilled water under ice cooling. When the sol-gel transition temperature of this aqueous solution was measured, it was found to be 20° C.

### Production Example 8

42.0 g of N-isopropylacrylamide and 4.0 g of n-butyl methacrylate were dissolved in 592 g of ethanol. To the resultant mixture solution, an aqueous solution which had been obtained by dissolving 11.5 g of polyethylene glycol dimethacrylate (PDE 6000, mfd. by NOF Corporation) in 65.1 g of water was added. The resultant solution was heated to 70° C. under a nitrogen stream. While the solution was maintained at 70° C. under a nitrogen stream, 0.4 mL of N,N,N′,N′-tetramethylethylenediamine (TEMED) and 4 mL of 10% ammonium persulfate (APS) aqueous solution were added to the solution, and then, the thus obtained solution was subjected to a reaction for 30 minutes under stirring. Further, 0.4 mL of TEMED and 4 mL of 10% APS aqueous solution were added thereto 4 times at 30-minute intervals, and the polymerization reaction was terminated. The reaction mixture was cooled to 5° C. or lower, it was diluted with 5 L of cold distilled water with a temperature of 5° C. Thereafter, the solution was concentrated to 2 L at 5° C., by using an ultrafiltration membrane with a molecular weight cutoff of 10×10⁴.

4 L of cold distilled water was added to the concentrated solution for dilution, and the above concentration operation using the ultrafiltration was conducted again. Thereafter, the above dilution and ultrafiltration concentration were repeated 5 times, so as to eliminate The product with a molecular weight of 10×10⁴ or lower. The product which had not been filtrated by the above ultrafiltration (product remaining in the ultrafiltration membrane) was recovered and freeze-dried, so as to obtain 40 g of the hydrogel-forming polymer (TGP-6) according to the present invention with a molecular weight of 10×10 or higher.

1 g of the thus obtained hydrogel-forming polymer (TGP-6) according to the present invention was dissolved in 9 g of distilled water under ice cooling. When the sol-gel transition temperature of this aqueous solution was measured, it was found to be 7° C.

### Production Example 9

45.5 g of N-isopropylacrylamide and 0.56 g of n-butyl methacrylate were dissolved in 592 g of ethanol. To the resultant mixture solution, an aqueous solution which had been obtained by dissolving 11.5 g of polyethylene glycol dimethacrylate (PDE 6000, mfd. by NOF Corporation) in 65.1 g of water was added. The resultant solution was heated to 70° C. under a nitrogen stream. While the solution was maintained at 70° C. under a nitrogen stream, 0.4 mL of N,N,N′,N′-tetramethylethylenediamine (TEMED) and 4 mL of 10% ammonium persulfate (APS) aqueous solution were added to the solution, and then was subjected to a reaction for 30 minutes under stirring. Further, 0.4 mL of TEMED and 4 mL of 10% APS aqueous solution were added thereto 4 times at 30-minute intervals, and the polymerization reaction was terminated. The reaction mixture was cooled to 10° C. or lower, it was diluted with 5 L of cold distilled water with a temperature of 10° C. Thereafter, the solution was concentrated to 2 L at 10° C., by using an ultrafiltration membrane with a molecular weight cutoff of 10×10⁴.

4 L of cold distilled water was added to the concentrated solution for dilution, and the above concentration operation using the ultrafiltration was conducted again. Thereafter, the above dilution and ultrafiltration concentration were repeated 5 times, so as to eliminate The product with a molecular weight of 10×10⁴ or lower. The product which had not been filtrated by the above ultrafiltration (product remaining in the ultrafiltration membrane) was recovered and freeze-dried, so as to obtain 22 g of the hydrogel-forming polymer (TGP-7) according to the present invention with a molecular weight of 10×10⁴ or higher.

1 g of the thus obtained hydrogel-forming polymer (TGP-7) according to the present invention was dissolved in 9 g of distilled water under ice cooling. When the sol-gel transition temperature of this aqueous solution was measured, it was found to be 37° C.

### Example 1

NK cells cultured in normal medium under normal gravity (a) and microgravity (b) for 6 hours and NK cells cultured in TGP medium of sol-gel transition temperature of 20° C. for 6 hours under normal gravity (c) and microgravity (d) were compared for cell population and NO level, and FACS analysis results.

Peripheral blood was collected from autoimmune enhancement therapy (AIET) patients as per the national regulatory guidelines, after informing them and with their consent. Separation of cells and culturing was carried out in a GMP conformant class 10,000 clean room. The collected peripheral blood was centrifuged at 3,000 rpm for 10 min at room temperature, the supernatant plasma and the precipitated hemocyte pellets were separately transferred to sterile centrifuge tubes. The plasma was inactivated by heat treatment at 56° C. for 30 minutes, and then centrifuged at 3,000 rpm for 10 minutes at room temperature. The supernatant plasma was placed in a sterile centrifuge tube and stored at −20° C. To separate the mononuclear cells, phosphate buffer (PBS) was mixed well with the hemocyte pellets that were placed in the sterile centrifuge tube, in a capacitance ratio of 1:1. In addition, a lymphoprep solution (Axis-Shield, Norway, Cat # LYS 3773) was overlaid on the hematocyte PBS mixture (i.e., 15 mL of lymphoprep solution and 30 mL of hematocyte PBS mixture) in a capacitance ratio of 1:2. The centrifuge tube was centrifuged in room temperature at 1,500 rpm for 10 min. The peripheral blood mononuclear cells (PBMNCs) were pipetted out and transferred to a sterile container so as not to disturb the separation layer. The PBS was added to the PBMNCs in a capacitance ratio of 2:1. After centrifuging at 1,200 rpm for 10 minutes in room temperature, the supernatant was discarded. The precipitated pellet was gently mixed with 30 mL of PBS. 20 ILL of PBS dispersion of the PBMNCs was taken out and the cell population was measured. The remainder was centrifuged at 1,200 rpm for 10 minutes in room temperature, the supernatant was discarded and the PBS was added and stored for cell culturing. The cell population was measured using trypan blue exclusion method. The PBMNCs was dispersed in a predetermined volume of Optimizer media ((Invitrogen, USA)) so as to achieve a cell density of 1×10̂6 cells/mL, cultured at 39° C. for 24 hours in a NK cell culturing flask (Biotherapy Institute, Tokyo) and thereafter at 37° C. for 24 hours under 5% CO2. The cells in the flask were centrifuged at room temperature of 1,200 rpm for 8 min along with the flask washing PBS solution. The NK cells were dispersed in Optimizer Basal Culture Medium ((Invitrogen, USA)) containing interleukin-2 ((Novartis Pharma AG, Basel, Switzerland)) and then cultured in a flask at 37° C. for 10 days˜14 days under 5% CO 2. Feeder layer or animal derived serum was never used in the entire process.

The NK cells were divided into normal medium group TGP medium group. In the normal medium group, approximately 20,000 NK cells, dispersed in ALyS 505 medium ((Cell Science & Technology Inst., Inc. Japan)) were placed in 2 mL sterile microcentrifuge tubes. In the TGP medium group, approximately 2,000 NK cells, dispersed in TGP medium were placed in 2 mL sterile microcentrifuge tubes. 1 g of freeze-dried TGP (Mebiol Gel, Mebiol Co., Ltd., Hiratsuka City) was dissolved in 10 mL of ALyS 505 medium, and the cells were dispersed in low-temperature sol state.

The normal medium group and TGP medium group were respectively divided into the normal gravity group and the microgravity group. The microgravity group was cultured at 37° C. for 6 hours in the microgravity mode ( 1/1000 G) in a microgravity environment cell culture equipment called Zeromo (CL-5000, manufactured by Kitagawa Iron Works, Ltd.), and the normal gravity group was cultured at 37° C. for 6 hours in normal environment.

Then, the viable cell population was measured in the collected NK cells, and measurement was conducted for the NO level and FACS analysis.

NO level was measured using Griess assay. Direct detection is extremely difficult as the half-life of the aqueous solution of NO is as short as 3-5 seconds, but the NO2 and NO3 oxides of NO are stable. Griess assay is the standard method of detecting NO2 in a culture supernatant by color reaction.

When comparing by absorbance in Griess assay, the NO level was (a) 0.090 under normal gravity and (b) 0.084 under microgravity in the normal medium. The NO level in TGP medium was (c) 0.126 under normal gravity and (d) 0.154 under microgravity. In other words, the NO level of NK cells had increased 1.7 times under the microgravity in TGP medium compared to the normal medium under normal gravity.

The cell population is the relative value, wherein the initial cell population is taken as 1. It was observed to be (a) 1.02 under normal gravity and (b) 1.32 under microgravity in normal medium, (c) 1.65 under normal gravity and (d) 1.27 under microgravity in TGP medium

TABLE 1
<Result of FACS analysis> (Example 1)

| | | Cell population | | |
|---|---|---|---|---|
| | | CD56 | CD16 | CD3 |
| | | Positive | Positive | Positive |
| | | cell | cell | cell |
| | | popula- | popula- | popula- |
| | | tion | tion | tion |
| Initial cell population | | 1 | 1 | 1 |
| Normal medium | Under normal gravity | 0.87 | 0.70 | 1.11 |
| (Cell population) | Under microgravity | 1.06 | 0.43 | 1.21 |
| TGP medium | Under normal gravity | 0.66 | 0.15 | 1.03 |
| (Cell population) | Under microgravity | 0.83 | 0.53 | 1.19 |

### Example 2

The cell population, NO level and FACS analysis results were compared for cord blood mononuclear cells (UCBMNCs) cells cultured in normal medium under normal gravity (a) for 6 hours, and under microgravity (b), and those cultured in TGP medium under normal gravity (c) and under microgravity (d) for 6 hours.

UCBMNCs were isolated from cord blood banks in India, from cord blood not used in transplantation medicine. Isolation and culturing of the cells was carried out in GMP conformant class 10,000 clean room. The collected peripheral blood was centrifuged at room temperature at 3,000 rpm for 10 minutes, the supernatant plasma was discarded and the precipitated hemocyte pellets were transferred to another sterile centrifuge tube. Phosphate buffer (PBS) was mixed well with the hemocyte pellets placed in a sterile centrifuge tube in a capacitance ratio of 1:1. Ficoll solution (GE Healthcare Life Sciences, UK, Catalog no. 17030010) was overlaid on the blood cell PBS dispersion in a capacitance ratio of 2:1 (Example, 15 mL of Ficoll solution in 30 mL of blood cell dispersion). After centrifuging at 1,500 rpm for 30 minutes at room temperature, the umbilical cord blood mononuclear cell layer was transferred to another sterile container without disturbing the isolation layer, and PBS which is twice the amount of the bone marrow mononuclear cell layer was added. After centrifuging at 1,200 rpm for 10 minutes at room temperature, the supernatant was removed with a pipette. 30 ml of phosphate buffer was added to the precipitated cells and the mixture was gently stirred. Next, 20 μl of the mixture was sampled and the cell population was measured. In the measurement of cell population, 20 μl of Turk's staining solution was added to 20 μl of the cell dispersion, 10 μl was then sampled and the viable cell population was measured. After 30 ml of the cell dispersion was centrifuged at 1,200 rpm for 8 minutes at room temperature, the supernatant was removed with a pipette.

UCBMNCs cells were divided into normal medium group and TGP medium group. About 20000 UCBMNCs cells dispersed in IMDM ((Gibco™, Life Technologies Corp, USA)) medium were placed in the normal medium group in 2 mL sterile microcentrifuge tubes. In the TGP medium group, TGP medium dispersed with approximately 20,000 UCBMNCs cells was placed in 2 mL sterile microcentrifuge tubes. 1 g of freeze-dried TGP (Mebiol Gel, Mebiol Co., Ltd., Hiratsuka City) was dissolved in 10 mL of IMDM and the cells were dispersed at low-temperature sol state. The TGP solution was allowed to gel at room temperature, and then overlaid with commercial human serum. After that, the cells were cultured at 37° C. under 5% CO2 environment for 7-14 days.

Normal medium group and TGP medium group were divided into normal gravity group and microgravity group, respectively. The microgravity group was cultured at 37° C. for 6 hours in the microgravity mode ( 1/1000 G) with Zeromo, a microgravity environment cell culture equipment (CL-5000, manufactured by Kitagawa Iron Works, Co., Ltd.). The normal gravity group was cultured at 37° C. for 6 hours in normal environment.

After that, the collected UCBMNCs cells were measured for viable cell population and NO level, and the FACS analysis was measured.

The NO level was measured using DAR (diaminorhodamine) assay. DAR assay is a method of using a fluorescent dye to directly detect NO in cells or supernatant. When relatively compared by fluorescence intensity, the NO level in normal medium was (a) (1.75) under normal gravity and (b) 2.69 under microgravity. In the TGP medium it was (c) 2.35 under normal gravity and (d) 3.10 under microgravity. That is, compared to normal medium and under normal gravity, the NO level of UCBMNCs cells had increased 1.8 times in the TGP medium under microgravity.

The cell population is the relative value, wherein the initial cell population is taken as 1. In the normal medium, it was (a) 1.17 under normal gravity and (b) 1.29 under microgravity. In TGP medium, it was (c) 1.17 under normal gravity, and (d) 1.29 under microgravity.

TABLE 2
<Result of FACS analysis> (Example 2)

| | | Cell population |
|---|---|---|
| | | CD34 Positive |
| | | cell population |
| Initial cell population | | 1 |
| Normal medium | Under normal gravity | 4.40 |
| (Cell population) | Under microgravity | 1.53 |
| TGP medium | Under normal gravity | 2.29 |
| (Cell population) | Under microgravity | 20.84 |

In other words, CD34 positive cell population of UCBMNCs cells had increased as much as 13.6 times in the TGP medium under microgravity compared to the normal medium microgravity.

### Example 3

The NO level and FACS analysis result were compared for bone marrow mononuclear (BMMNCs) cells cultured in normal medium for 6 hours under normal gravity (a) and under microgravity (b), and those cultured in TGP medium for 6 hours under normal gravity (c) and microgravity (d). Bone marrow harvested from a human was placed in a sterile centrifuge tube and centrifuged at 3,000 rpm for 10 minutes at room temperature; the supernatant was then removed with a pipette. 15 ml of precipitated cells were dispersed by adding 15 ml of phosphate buffer (Manufactured by Life Technologies, USA, Catalog no. 70011-044), and 15 ml of Ficoll solution (manufactured by GE Healthcare Life Sciences, UK Catalog No. 17030010) and overlaid. After centrifuging at 1,500 rpm for 30 minutes at room temperature, the bone marrow mononuclear cell layer was transferred to another sterilization container without disturbing the isolation layer, and phosphate buffer solution, which is twice the amount of bone marrow mononuclear cell layer, was added. After centrifuging at 1,200 rpm for 10 minutes at room temperature, the supernatant was removed with a pipette. 30 ml of phosphate buffer was added to the precipitated cells, the mixture was gently stirred, 20 μl of the mixture was sampled and the cell population was measured. In measuring the cell population, 20 μl of Turk's stain solution was added to 20 μl of the cell dispersion, 10 μl was sampled and the viable cell population was measured. The cell dispersion was centrifuged at 1,200 rpm for 8 minutes at room temperature, and the supernatant was removed with a pipette.

Thereinafter, the same experiment as in Example 2 was carried out, and the NO level and FACS analysis were measured for the BMMNCs cells.

When NO levels were measured in DAR assay and relatively compared by the fluorescence intensity, the NO level in normal medium was (a) 0.95 under normal gravity and (b) 0.78 under normal gravity; in the TGP medium, it was (c) 0.87 under normal gravity and (d) 2.90 under microgravity. In other words, the NO level of UCBMNCs cells was found to have increased 3.1 times under TGP medium microgravity than under normal gravity in normal medium.

TABLE 3
<Result of FACS analysis> (Example 3)

| | | Cell population |
|---|---|---|
| | | CD34 Positive cell |
| | | population |
| Initial cell population | | 1 |
| Normal medium | Under normal gravity | 0.59 |
| (Cell population) | Under microgravity | 1.71 |
| TGP medium | Under normal gravity | 0.78 |
| (Cell population) | Under microgravity | 1.57 |

That is, the CD34 positive cell population in BMMNCs cells had increased 2.7 times under TGP medium microgravity as compared to normal medium normal gravity.

### INDUSTRIAL APPLICABILITY

As specifically explained above, here, it is possible to efficiently enhance the function of NK cells, and has substantial utility in the treatment of solid organ cancer, treatment of blood cancers, and in the treatment of patients who are bone marrow transplant recipients

This method of regeneration can be very effectively used as the method of cell recovery, restoration and revival in regenerative medicine.

1. A method of enhancing functions of cells by embedding them in an aqueous solution containing a novel thermo reversible gelation polymer which at low temperature is in liquid state and at higher temperature is in gel state and by subjecting and culturing the cells under such embedded conditions to microgravity.
